(19) United States
(12) Patent Application Publication
(10) Pub. No.: US 2012/0203188 A1
(43) Pub. Date: Aug. 9, 2012

(54) MEDICAL IMAGING SEPTUM AND ACCESS PORT DEVICE AND METHOD

(76) Inventors: Christopoher N. Magalich, Apolo, PA (US); Chun Kee Lui, Apolo, PA (US)

(21) Appl. No.: 13/368,715

(22) Filed: Feb. 8, 2012

Related U.S. Application Data
(63) Continuation of application No. 12/783,909, filed on May 20, 2010, now abandoned.

(51) Int. Cl. A61M 5/00; B28B 5/00

(52) U.S. Cl. 604/288.02; 264/40.1

(57) ABSTRACT

Access, or injection ports, are disclosed. These are shown with various needle-penetrable sealing septums. The septum design is shown with a first and second (and optionally more) elastomeric regions. Indicia may be formed in at least one of said regions, by voids or otherwise and medical imaging additive may be used to provide radiopaque or other contrast between said regions to provide indicia. Such elements may include where two or more such regions are over-molded. The indicia may be on or embedded in the septum. Methods of making such septums and/or ports, including blending and molding is disclosed.

### CROSS-REFERENCE TO RELATED APPLICATION

This application is a continuation of application Ser. No. 12/783,909, filed May 20, 2010, which is hereby incorporated by reference.

### BACKGROUND

The present invention relates generally to injection or access ports, and more specifically to septum used in connection with such ports.

Injection ports, also known as access ports, are well known. They come in a wide variety of sizes, shapes, and styles, including excorporeal and subcutaneous. Uses for injection ports are for repeat injections and/or withdrawals of fluids into or out of the body.

Some injection ports have a elastomeric septum, and by having a needle that may be penetrated through the septum, the septum may re-seal in a substantially fluid type manner upon withdraw of the needle.

Often, one or more catheters may be attached to the port, such as to provide fluid access to one or more chambers inside the port.

Some injection ports are designed using radiopaque contrast media, including indicia, to identify the location of and/or to identify attributes of the injection port. For example, this may involve printing of radiopaque material and/or the use of foil as radiopaque material at the base of the port.

Some ports are made of metal, and some of plastic or both.

The present invention is new and different. While its septum(s) may be used in the aforementioned types of ports and others, the septum configuration is different. Such differences are set forth in the claims below.

### SUMMARY

The present inventions are defined by the claims, and only the claims. As set forth in the claims, such inventions may include the optional elements of an access port for subcutaneous use in a patient. Such elements may include a needle-penetrable sealing septum for an access port. It may have, a first and second (and optionally more) elastomeric regions. Indicia may be embedded in the septum. Indicia may be formed in at least one of said regions, and medical imaging additive, such as for example, radiopacity additive, may be used to provide contrast between said regions to provide indicia. Such elements may include where two or more such regions are over-molded.

The present inventions may include a method of making a septum and/or port, including blending and molding as claimed.

Further forms, objects, features, aspects, benefits, advantages, and embodiments of the present invention are enabled from the written description and drawings provided herewith.

### BRIEF DESCRIPTION OF THE DRAWINGS

FIG. 1 is a top plan view of an element or region of one example of the present invention.

FIG. 2 is a cross-sectional view taken along lines 2-2 of FIG. 1.

FIG. 3 is a bottom plan view of the element of FIG. 1.

FIG. 4 is a top plan view of one example of the present invention.

FIG. 5A is a cross-sectional view taken along line 5A-5A of FIG. 4.

FIG. 5B is a cross-sectional view of an alternative of the device of FIG. 5A.

FIG. 5C is a cross-sectional view of an alternative of the device of FIG. 5A.

FIG. 5D is a cross-sectional view of an alternative of the device of FIG. 5A.

FIG. 5E is a cross-sectional view of an alternative of the device of FIG. 5A.

FIG. 5F is a cross-sectional view of an alternative of the device of FIG. 5A.

FIG. 5G is a cross-sectional view of an alternative of the device of FIG. 5A.

FIG. 6 is a bottom plan view of the device of FIG. 4.

FIG. 7 is a top perspective view, partially cut away, of the device of FIG. 4.

FIG. 8 is a flow chart of one example of a method of the present invention.

FIG. 9 is a side cross sectional view of the device of FIG. 4 in one example of a port.

### DESCRIPTION OF THE SELECTED EMBODIMENTS

For the purpose of promoting an understanding of the principles of the invention, reference will now be made to the embodiments illustrated in the drawings and specific language will be used to describe the same. These are merely examples. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

As used in the claims and the specification, the following terms have the following definitions:

The terms “access port” or “injection port” mean a port that provides repeated access to a medical patient's blood vessels or other body vessels or cavities for purposes of adding and/or withdrawing fluids.

The term “additive” means one or more ingredients blended, embedded, added or formed into and which increase contrast and/or opacity for medical imaging. By way of example only, and without limitation, this may include barium compounds, barium sulfate, iodine compounds, tungsten, titanium, palladium, other metals (particles (including powders) and otherwise), compounds and/or blends thereof, bubbles, particulate, combinations thereof, and otherwise.

The term “alpha-numeric shape” means a structure in the shape of one or more letter, number (Arabic, roman, or otherwise), or both, alone or in combination with other shapes, symbols, and/or targets.

The term “barium sulfate” means BaSO₄, as well as any compound, mixture, combination or blend containing BaSO₄.

The term “blending” means to mix, react, dissolve, suspend and/or otherwise combine, mechanically, chemically or otherwise.

The term “catheter” means flexible medical tubing. It may include or not include radiopaque marking. It may include or not include reinforcement; it may include or not include other structures or features attached to it. It may have one, two or more lumens.

The term “chamber” means a three-dimensional space.

The term “circumferential rib/groove interface” means one or more structures that contact to hold, or help hold, two or more elements together, with such structures projecting (male structure) and/or receiving (female structure) generally around an edge or circumference. Those structures may be intermittent, continuous, or otherwise. They may alternate, with, for example both male and female elements on one or both elements. They may be located on the top, side, bottom, inside and/or outside of one or both elements.

The term “clear” means transparent, or at least translucent (whether or not color tinted), sufficient to allow human visualization of the indicia.

The term “conduit” means a lumen or passageway through a structure sufficiently large to allow the passage of fluids.

The term “contrast” means visual distinguish-ability in terms of imaging as between two or more regions and/or indicia due to relatively higher and lower opacity.

The terms “cured” and “curing” mean the toughening or hardening of a polymer and/or elastomer material by cross-linking of polymer chains, normally brought about by chemical additives, ultraviolet radiation, electron beam, heat, cooling, and/or a combination thereof.

The terms “elastomeric” and “elastomer” and “elastomeric material” mean a polymer, or including a polymer having notably low Young's modulus and high yield strain, or rubbery as compared with other rigid materials such as metal or wood. It is elastic, and may, but does not necessarily include viscoelastic material. It may include thermoset polymers and thermoelastic polymers and otherwise, as well as a blending thereof. Silicon rubber, silicone, latex, or other materials, alone, blended and/or as co-polymers, are but examples that may be used or included.

The term “embedded” means closed in or surrounded, wholly or at least partially, within one or more regions of material. This includes layers embedded in or between other layers, particles embedded in elastomeric or other material, foil or printing embedded in or between layers, and/or otherwise.

The terms “flowed” and “flowing” mean movement of a liquid from one location into another location.

The term “fluid sealing engagement” means contact between two or more elements that substantially stops or restricts fluid flow therebetween.

The term “forming indicia” means molding, cutting, stamping, dissolving, machining, printing, coating, glazing, spraying, extracting and/or otherwise creating indicia or the space or profile around and defining indicia.

The term “housing” means a structure which directly and/or indirectly defines, surrounds and/or supports one or more chambers therein.

The term “human visualization” means reading and/or being able to be read using visible light.

The term “in an amount greater than” is a relative term meaning that one region has a greater aggregate quantity of something (such as for example radiopaque material) when measured in a vertical (Z-axis) direction than other region or regions.

The term “indicia” means a shape that is human and/or machine readable as symbolic of something, such as for example a number, letter, bar code, RFID, trademark, symbol, target and/or combination thereof. Indicia may be three dimension and/or essentially two-dimensional, and/or essentially two-dimensional but lying in a curved plane.

The term “integral” means made from the same piece of material, such as for example, commonly molded in the same piece of plastic and/or commonly machined in the same piece of metal.

The term “MRI” and “magnetic resonance imaging” means creating an image or visualization using magnetic waves, including without limitation, and MRI machine, nuclear magnetic resonance imaging, or otherwise.

The term “medical imaging” means creating an image or visualization using sonography imaging, MRI and/or radio imaging.

The term “medical imaging media” means a substance (solid, liquid, cured, dried, elastomeric, foil, additive, particle, powder, ink, three dimensional, essentially two dimensional and/or otherwise) which provide medical imaging contrast.

The term “member” means a solid (including cured) structure made of one or more pieces. It may be rigid, flexible, or both.

The term “molding” means to cure a liquid in a mold.

The term “needle access” means adapted to receive a medical needle to add and/or withdraw fluids.

The term “needle-penetrable sealing septum” means a barrier which is penetrable by a medical needle, which forms a fluid sealing engagement around the needle during sealing and provides a seal against any substantial fluid flow after withdraw of the needle.

The term “occupied” means to reside in a space, void or region.

The terms “optional” and “optionally” mean not required. Moreover, the lack of the use of the term optional or optionally does not imply that the feature is required, but rather the use of optional and optionally are merely to stress that aspect.

The term “over-molded” means to cure one or more regions in a mold while in contact with a previously cured (partially or wholly cured) region(s) and/or with other(s) structures or layers in the mold.

The term “plan profile” means the shape or area or profile when viewed from a top plan perspective (down along a Z-axis).

The term “radio imaging” means creating an image or visualization using electromagnetic spectrum waves, including without limitation, fluoroscopy, x-rays, or otherwise.

The terms “radiopaque” and “radiopacity” mean stopping or impeding electromagnetic waves (for example X-rays, fluoroscopy) from radio imaging such as to be visible via radio imaging.

The term “region” means a three-dimensional part or entirety of a structure or element. Unless specifically noted otherwise as an “essentially two-dimensional region”, “region” does not include a very thin layer such as printing or foil.

The terms “sonography imaging” and “sonography” mean creating an image or visualization using sound waves, including without limitation, ultrasound, or otherwise.

The term “subcutaneous” means under the skin of a patient.

The term “substantially covers” covering at least 80% of an underlying structure when viewed from a plan profile.

The term “substantially filled” means occupying at least 80% of a former void with a solid and/or liquid.

The term “void” means substantially absent a solid or liquid.

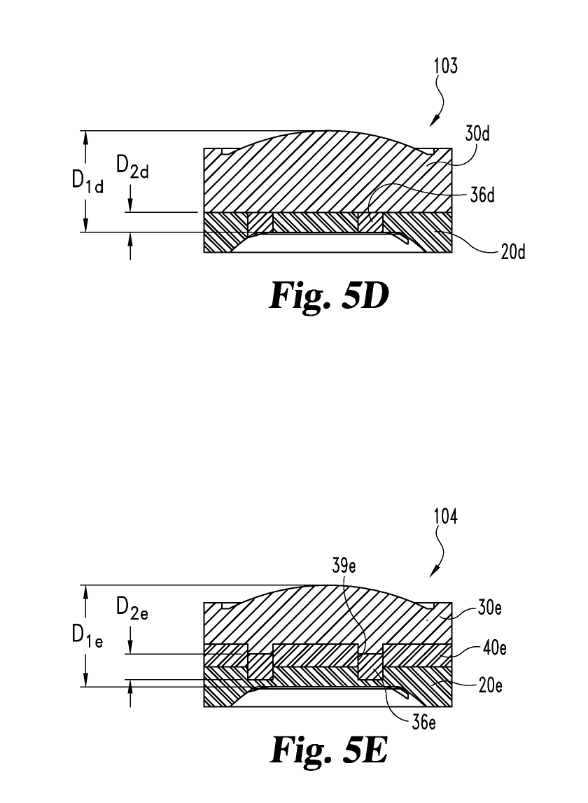

The term “Z-axis” means a vertical axis. For example, in FIG. 5A, which shows a section in an X-Z plane, the Z-axis would appear as a vertical line therein.

Articles and phases such as, “the”, “a”, “an”, “at least one”, and “a first”, are not limited to mean only one, but rather are inclusive and open ended to also include, optionally, two or more of such elements.

The language used in the claims and the written description is to only have its plain and ordinary meaning, except as explicitly defined above. Such plain and ordinary meaning is inclusive of all consistent dictionary definitions from the most recently published (on the filing date of this application) general purpose Webster's dictionaries and Random House dictionaries.

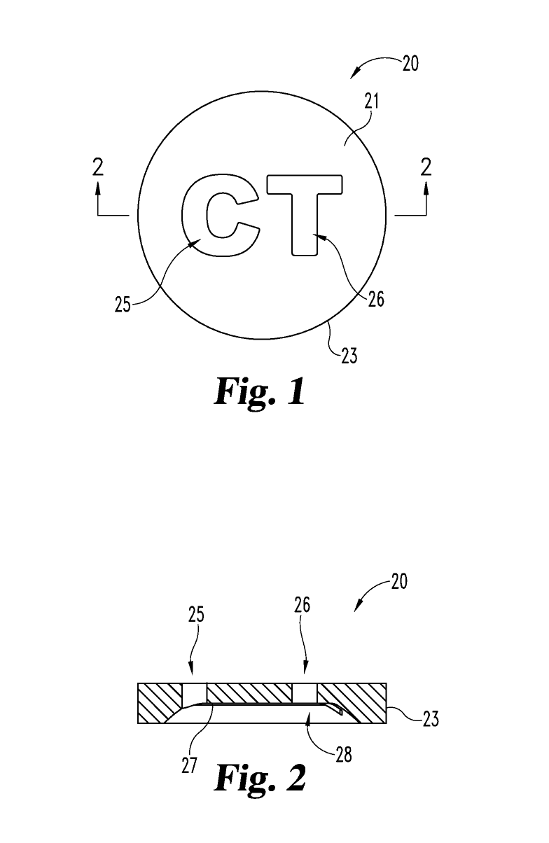

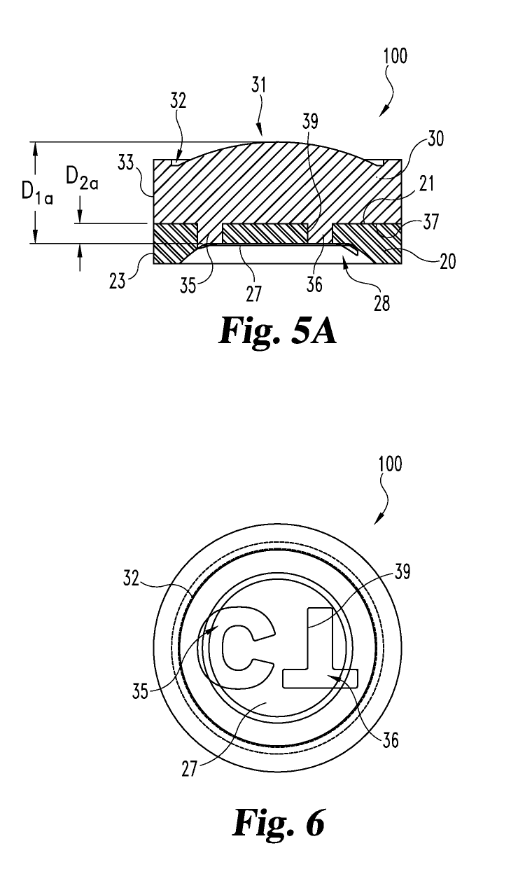

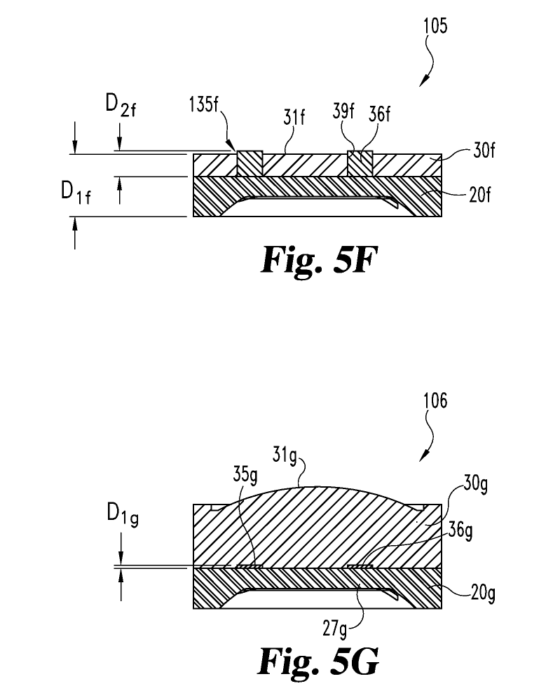

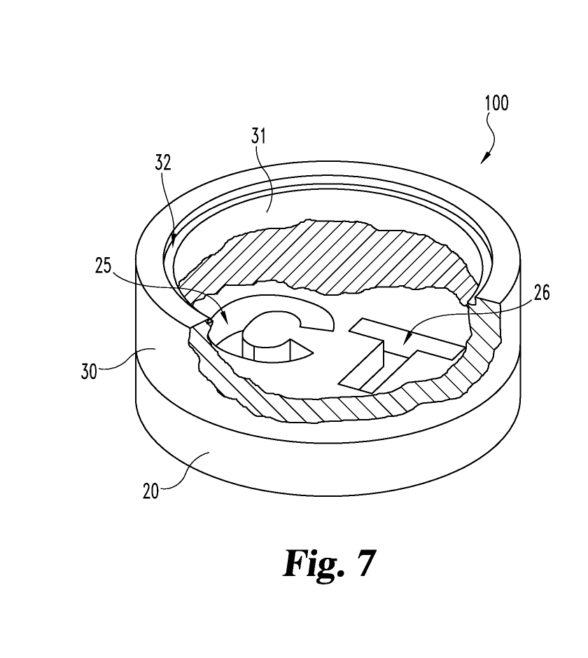

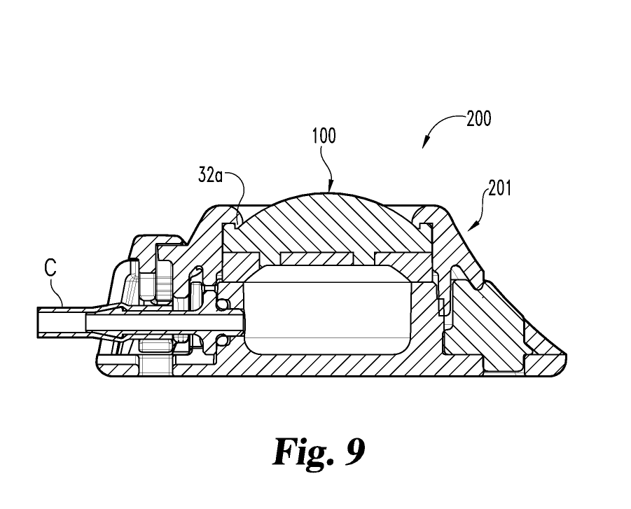

Referring FIGS. 1-7, devices 100, 101, 102, 103, 104, 105 and 106 are shown comprising a needle-penetrable sealing septum for an access port. Any type of access port may be employed, and port 300 (see FIG. 9) is illustrated as merely one example. The device(s) may comprise a first elastomeric region 20 or 30, a second elastomeric region 30 or 20 (contra to the aforementioned first region). Preferably, the septum and the indicia thereon or therein is needle-penetrable without preventing one or more elastomeric region providing a seal against any substantial fluid flow through the sealing septum after withdraw of the needle.

There may be indicia formed at least in part as an indicia void in at least one of said regions. Such indicia may, for example, be letters and/or numbers and/or other symbols. Only by way of example, voids 25 and 26 shown, for example as the letters “C” and “T” respectively (or “CT” collectively). In such case, the indicia appear as “C” and “T”. Other indicia may be used, such as for example, “15” or “15 mL” to indicate the flow rate rating of the port (such as 15 milliliters per minute). Other letters and numbers may be used. The indicia may be or also include trademarks, logos, and targets for needle aiming and/or otherwise. The indicia may be co-planer, or optionally lie along a vertical Z-axis in two or more planes to provide 3-dimentional perspective and/or orientation under radio imaging. Such 3-dimentionality may also be provided by combining indicia of the present invention with indicia on other parts of the port, such as within the septum and/or such as on the port's base. The void(s) may be occupied at least in part by the other of said regions. Thus, for example with reference to FIG. 5A, region 30 occupies the voids 25 and 26, respectively, with indicia 35 and 36. Optionally, this could be inverted for one or more of such regions. As example, a void could be formed in region 30 occupied by part of region 20.

Additive for medical imaging is normally included substantially throughout elastomeric material in one of said regions 20 or 30, normally in an amount greater than the other of said regions. Additive may be in region 20, or in region 30, or both, or other regions, indicia or layers described below. Normally, the amount of additive, when used, is different as between such regions. This may provide contrast between said regions to provide indicia. This may, for example comprise a differential in depth (and/or depth taking into account relative additive density) between one region and other region(s) and/or the overall septum. Such height differential is shown in various examples, such as in FIG. 5A the differential between Height D₁ₐ and D₂ₐ, in FIG. 5B the differential between Height D_{1b} and D_{2b}, in FIG. 5C the differential between Height D_{1c} and D_{2c}, or in FIG. 5D the differential between Height D_{1d} and D_{2d}, or in FIG. 5E the differential between Height D_{1c} and D₂ₑ, or in FIG. 5F the differential between Height D_{1f} and D_{2f}. Also, such differential may be expressed without comparison of depths between regions, but rather merely as the depth of one region, such as for example depths D₂ₐ in FIG. 5A, D_{2b} in FIG. 5B, D_{2c} in FIG. 5C, D_{2d} in FIG. 5D, D₂ₑ in FIG. 5E, D_{2f} in FIG. 5F, or D_{1g} in FIG. 5G. Or, this may be expressed as the height of which ever layer(s) or region(s) have the additive, such as for example in FIG. 5F, rather than depth D_{2f} of region 36f, instead the contrast additive could optionally be in region 30f, in which case the depth (or Z-axis thickness) of region 30f could be used. The same could be true with respect to other layers or regions, such as merely for example region 40e, 20e, 30e, 20d, 30d, 20c, 30c, 20b, 30b, or otherwise. With such depths and/or differential(s), the presence of medical imaging opacity additive in region 20 but not in region 30 (or at least less in region 30)(or vice-versa, namely additive in region 30 but not 20) may provide contrast. Such contrast may occur at interfaces between regions, such as, for example, interface 39 (FIG. 5A), 39b (FIG. 5B), or 39e (FIG. 5E) or others. Instead or in addition to the aforementioned depth differential, contrast may be provided or enhanced by variance or differential in density and/or quality of the opacity additive and/or localization of it near, around and/or to form the indicia. This may be done during molding. Or, optionally for example, indicia 36 (FIG. 5A), 36d (FIG. 5D), 36e (FIG. 5E), 36f (FIG. 5F) or 36g (FIG. 5G) or others could be coated, printed, glazed, encased, on some or all of its outside surfaces with medical imaging media.

Any of the indicia may be located in the top, middle and/or bottom of the depth of the septum, including optionally throughout all, most or only some of its depth.

Optionally, the void 25 and/or 26 (see e.g. FIGS. 1-3) may include an alpha-numeric shape.

Optionally, the void(s) in a first region 20 or 30 may be filled partially or even substantially filled with elastomeric material from the other of said regions. Examples of such filling may include one or more of 35 and 36 in FIG. 5A, 36b in FIG. 5B, 36c in FIG. 5C, 36d in FIG. 5D, and 36e in FIG. 5E. These may be modified, combined, mixed and/or matched.

Optionally, the elastomeric material such as that forming 35, 36, 36b, c, d, e, f, and/or g may be flowed into the void and thereafter cured in that void.

Optionally, the aforementioned second region, be it 20 or 30, may be over-molded over the first region.

Optionally, such over-molding may result in bonding between the two regions.

Also, optionally wherein the second region may substantially cover said first region (see e.g. FIG. 5A-G) or not, and the first and second regions may be molded together, or not, with respect to each other.

Optionally, the additive may be any of one or more materials. As but one example, such additive may include barium sulfate. It also could include that blended with other radiopacity additive(s), and/or others.

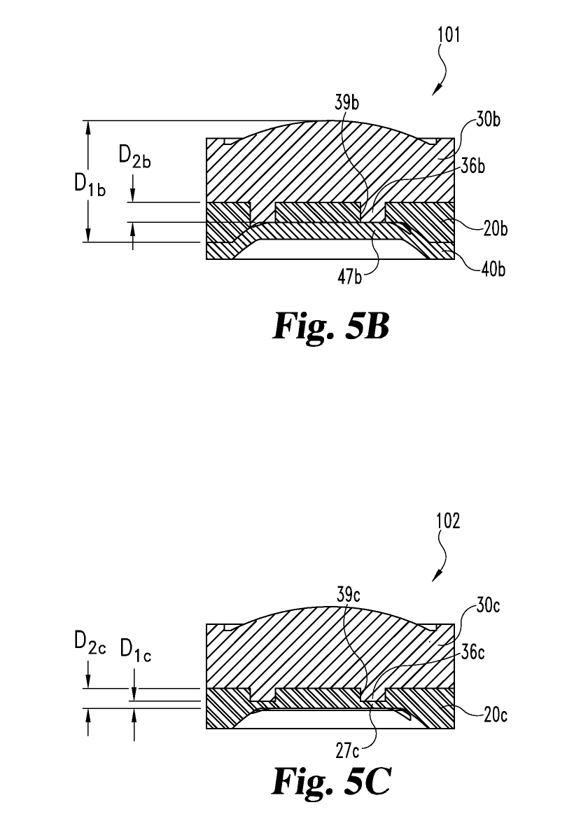

Optionally, the septum may be generally round in plan profile (see FIG. 4), and further may optionally comprising a circumferential rib/groove interface, such as for example a rib/groove interface between groove 32 (see FIG. 5A) and rib 32a (see FIG. 9) for fluid sealing engagement with an injection port. It could also be other shapes, such as oval, square, “8” shaped, triangular, and otherwise.

Optionally, and indeed normally, the septum is in combination with an access port housing. Many shapes and styles may be used, and one example is shown in FIG. 9 with septum 100 housing 201 collectively making up port 200.

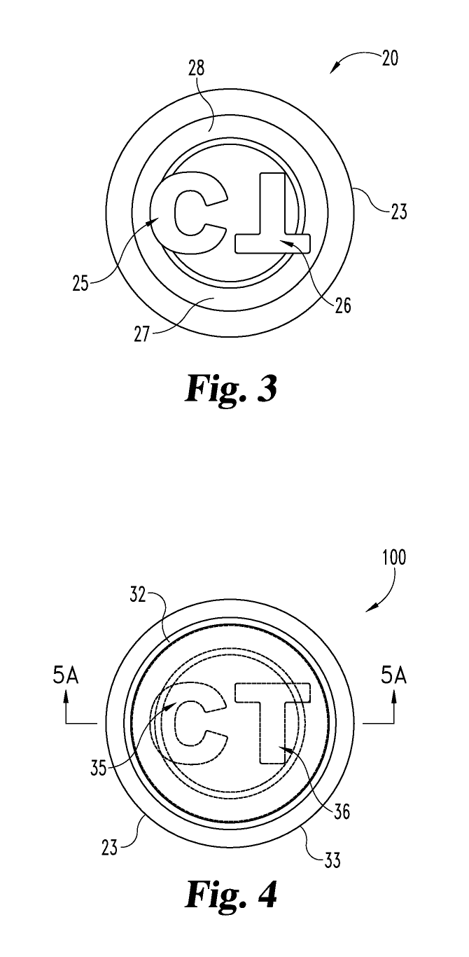

The septum may take wide variety of shapes, styles, sizes and configurations. It may also have a wide variety of features or not. For example, as seen in FIG. 2, optionally a bottom recess 28 may be provided defined in part by bottom surface 27 of region 20. Recess 20 may, or optionally may not, be in communication with recesses, such as recess 25 or 26 (see FIG. 2).

Also, optionally, the septum may be modified from the example illustrated in FIGS. 1-4, 5A and 6. For example, such alternatives may be seen by comparing FIG. 5A with, for example, the versions of septum 101, 102, 103, 104, 105 and/or 106 in FIGS. 5B, 5C, 5D, 5E, 5F and 5G, respectively. Note that in those alternatives, similar reference characters as previously described correspond to like structure. Thus, for example, FIG. 5B, the reference characters ending in “b” correspond to the components in FIG. 5A without such “b” on the end of the reference character. For example, region 30b corresponds with region 30. Interface 39b corresponds with interface 39. Similarly, with the other FIGS. 5C, 5D, 5E, 5F and 5G the reference characters have the suffix “c”, “d”, “e”, “f” or “g”, respectively. Other optional variations are discussed further below.

For example, in FIG. 5B, an optional third region 40b is illustrated. It may, for example, at bottom region 47b, cap the void filled with indicia 36b. It may be manufactured in any number of ways. For example, region 40b may be first molded, with region 20b over-molded thereover, and with region 30b molded thereover. Optionally, the same manufacturing step may be used, except that region 20b is separately molded, and dropped in on top of region 40b or otherwise. When the indicia includes 36b, such indicia is shown in contact with region 40b and region 20b. It is also shown embedded in septum 101.

FIG. 5C shows a variation in which two regions are present, region 20c and region 30c. In this case, the void which will provide for the formation of indicia does not pass all the way through region 20c, but rather is capped with bottom region 27c. In such case, if region 30c is over-molded over region 20c, material is flowed into the void where indicia 36c is formed. Yet, due to the presence of cap bottom region 27c, such flow is contained. When the indicia includes 36c, such indicia is shown in contact with region 20c. It is also shown embedded in septum 102.

FIG. 5D shows a version similar to FIG. 5A, except that indicia 36d is not integral with region 30d, but rather is separate. As illustrated in FIG. 5D, region 36d, which may comprise the indicia, is shown as a solid material, often an elastomeric region. For example, it may include radiopacity additive. Alternatively region 36d may lack, or have less, radiopacity additive or other attributes as compared to other regions, such as for example region 20d. Thus, for example, region 36d may lack radiopacity, whereas region 20d may have radiopacity, such as by an additive. When the indicia includes 36d, such indicia is shown in contact with region 30d and region 20d. It is also shown embedded, and indeed partially embedded in septum 103. Such is the case with indicia 36 as exemplified in FIG. 5A. Further still, indicia 36d may be, rather than a solid material (an elastomer or otherwise) simply a void (void, air, gas, or may comprise some other fluid such as a liquid). In such case, typically, it would be capped such as illustrated in FIGS. 5C and/or 5E. Thus, such void may be present while other regions, such as region 20d have a radiopacity additive.

FIG. 5E shows septum 104 having four regions, rather than the previously mentioned two or three regions. Optionally, more than four regions may be provided as well. This particular example, region 36e corresponds with the indicia. It may have or not have the attributes previously described with respect to indicia 36 and/or indicia 36d. This can be seen, such region 36c is combined with region 20e, region 40e and region 30e. When the indicia includes 36e, such indicia is shown in contact with regions 20e, 30e and 40e. It is also shown embedded, and indeed fully embedded in septum 104. As before, although illustrated as a solid material, such as an elastomer, or otherwise, region 36e may also be a void.

FIG. 5F shows regions 20f, 30f, and 36f. As before more layers or regions may optionally be included. Also, optionally region 36f could be integral with region 20f, although the drawing shows a version where they are not integral. Septum 105, like the others, may have any shape and is shown here with a flat top surface 31f, rather than a domed, concave or other surface. Contrast, as before, may occur at many locations, edges and/or surfaces, such as interface 39f Region 36f in this example comprises indicia. It is show protruding above top surface 31f, forming a tactile rise 135f. This optionally provides for palpation by the user in subcutaneous applications. Other ribs, bumps or other such rises may optionally be formed or provided in any of the septums 100-106 or otherwise disclosed herein. Thus, for example here in FIG. 5F, region 36f may both have medical imaging additive therein, and serve the dual purpose of providing for locating the septum and/or port by palpation. When the indicia includes 36f, such indicia is shown in contact with region 30f and region 20f. It is also shown embedded, and indeed partially embedded in septum 105. Note that septum 105 is shown as not having a rib/groove interface, which, like the other septums may optionally be included or excluded. Also, it is shown as having a recess on the bottom, but optionally this too may be excluded in FIG. 5F and the other examples.

FIG. 5G shows septum 106 having indicia at 35g and 36g. The indicia in that example has depth D_{1g}. This indicia may be printing and/or foil or other thin layer of medical imaging media. Such indicia is shown in contact with region 30g and region 20g. It is also shown embedded, and indeed fully embedded in septum 106. Optionally, it may be applied and in contact with (by printing, screening, spraying or otherwise) to one or more outer surfaces, such as top surface 31g and/or bottom surface 27g (facing and partially defining a chamber in the port) of one or more regions, instead of, or in addition to, being embedded in septum 106. This may be made, merely by way of example, by forming (molding or otherwise) one region, placing or printing indicia 36g in contact therewith, and then over-molding the other region. Optionally, instead regions 20g and 30g may be integral, such as by common or concurrent molding, and with optional fusing or curing together as an elastomeric monolith or integral member. As with the other examples of septums, such indicia is needle-penetrable without preventing said first elastomeric region (20g and/or 30g, alone or in combination) providing a seal against any substantial fluid flow through the sealing septum after withdraw of the needle.

Any of the aforementioned indicia may be as a negative, rather than as a positive, namely that some of all of the indicia is made by forming the space or profile around and defining indicia. For example, the indicia may comprise a medical imaging media around the shape of a letter and/or number.

Any of the above regions, whether two, three, four or more, may be formed by over-molding or formed by other techniques, or a combination thereof. For example, an initial region may be molded, followed by a second region which is over-molded. Alternatively, or in addition to that, there may be a region that is molded, and in a pre-molded, cured region set in place, and then a third region over molded on that assembly. Typically, when over molding, regions bond together by virtue of the over molding. This may include, but does not require, for example over-molding over printing or foil 36g. Optionally, adhesives, mechanical locking structures and otherwise may form or assist such holding of the regions together.

Optionally, one or more regions may be clear. For example, a top region (such as for example regions 20, 20b, 20c, 20d, 20e, 20f and/or 20g), and/or other regions could be clear. This may allow human visualization of indicia, when such indicia is below or embedded in such region. Such human visualization may be in addition to medical imaging.

The various features and/or structures of the foregoing examples may be mixed and/or combined.

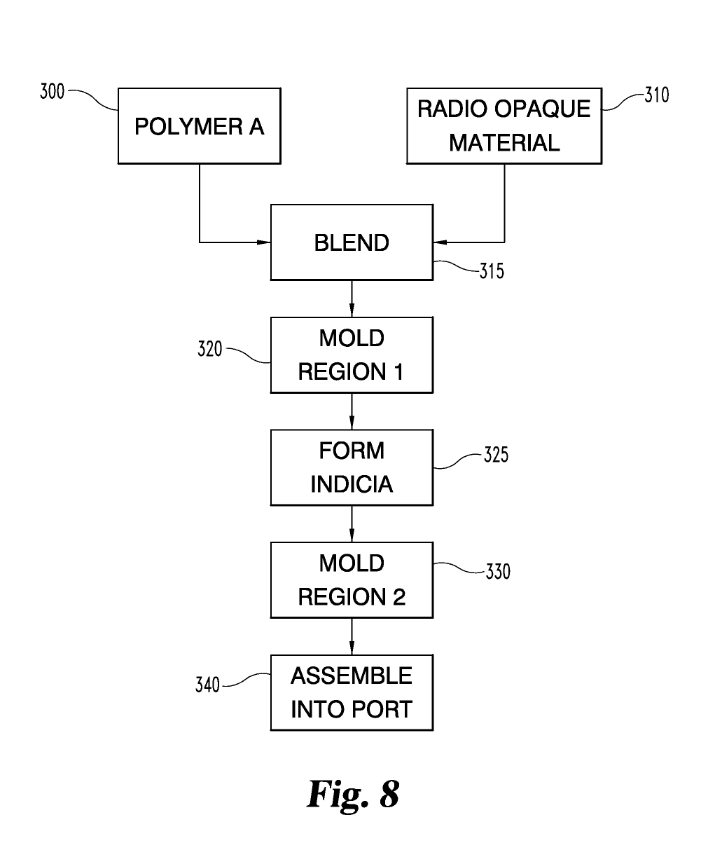

How the device is manufactured is not necessarily critical. However, FIG. 8 diagrams merely one example of a method of manufacture. In such case, polymer A, 300, and a radiopaque (and/or other non-radiopaque medical imaging media) material or additive 310 may be blended at 315. Concurrently, although preferably thereafter, a first layer at 320 is molded. Separately, or as a part of 320, forming of an indicia profile may be done at 325. Preferably, such forming of indicia profile is done by virtue of a pre-existing shape in the mold used in act 320 of molding. Thus, for example, an alpha-numeric positive mold (or negative mold) may form part of the shape of the mold used in molding step 320, with the removal thereof providing such indicia (by void or otherwise) upon separation of that mold. Thereafter, particularly if the septum is made by over-molding, there may be a second mold layer to step 330. As mentioned, this may be done by over-molding the byproduct of acts 320 and/or 325, but optionally also might include over-molding on a pre-cured element that has been stamped, cut or otherwise formed prior to the molding act 330. Optionally, molding act 330 may be done with a different mold or mold half. Thus, for example, in connection with FIG. 5D, an initial mold used in connection with act 320 might have the top profile of merely region 20 (see FIG. 2). Thereafter, such top mold is removed, and then for molding act 330 a different mold is used to shape the top surface of region 30, including for example, its optional domed surface 31 (see FIG. 5A).

Thereafter, optionally, it may be assembled into a port. As mentioned, merely one such example is shown in FIG. 9. Such assembly may include, but does not necessarily require, a engagement, such as a rib/groove engagement circumferential or otherwise. Other such assembly may simply include adhesive, encasement within the port, interlocking projections, friction fit and otherwise. Optionally, as set forth in one or more claims, the septum may have one region over-molded on another region but without indicia. Normally, however, indicia would be present on or in the septum. Note also that such septums according to the present invention may optionally be included in ports having two or more septums, ordinarily with two or more corresponding chambers.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

What is claimed is:

1. A device comprising a needle-penetrable sealing septum for an access port, comprising:
at least a first elastomeric region;
medical imaging indicia that is in contact with said first elastomeric region of the sealing septum, wherein said indicia is needle-penetrable without preventing said first elastomeric region providing a seal against any substantial fluid flow through the sealing septum after withdraw of the needle.

2. The device of claim 1, wherein said indicia is at least partially embedded in said first elastomeric region.

3. The device of claim 2, and further comprising at least a second elastomeric region, wherein said second region is over-molded in contact with said first region.

4. The device of claim 3, and further comprising:
a void in at least one of said regions;
wherein said void is substantially filled with elastomeric material from the other of said regions; and,
radiopacity particle additive substantially throughout elastomeric material in one of said regions in an amount greater than the other of said regions, providing radiopaque contrast between said regions to provide radiopaque indicia.

5. The device of claim 4, wherein elastomeric material is flowed into said void and thereafter cured in said void, and wherein said second region substantially covers said first region and said first and second regions are molded together with respect to each other.

6. The device of claim 5, wherein said indicia comprises an alpha-numeric shape and is fully embedded within the septum.

7. The device of claim 6, wherein said radiopacity additive includes barium sulfate.

8. The device of claim 7, wherein the septum is generally round in plan profile, and further comprising a circumferential rib/groove interface for fluid sealing engagement with an injection port.

9. The device of claim 8, further comprising the septum in combination with an injection port housing.

10. The device of claim 9, wherein at least one region is clear allowing human visualization of said indicia through said clear region.

11. The device of claim 1, and further comprising at least a second elastomeric region, wherein said second region is over-molded in contact with said first region.

12. The device of claim 11, and further comprising:
a void in at least one of said regions;
wherein said void is substantially filled with elastomeric material from the other of said regions; and,
particle additive substantially throughout elastomeric material in one of said regions in an amount greater than the other of said regions, providing medical imaging contrast between said regions to provide indicia.

13. The device of claim 12, wherein elastomeric material is flowed into said void and thereafter cured in said void, and wherein said second region substantially covers said first region and said first and second regions are molded together with respect to each other.

14. The device of claim 1, wherein said indicia comprises an alpha-numeric shape and is fully embedded within the septum.

15. The device of claim 11, wherein said indicia comprises an alpha-numeric shape and is fully embedded within the septum.

16. The device of claim 1, wherein said indicia includes barium sulfate.

17. The device of claim 1, wherein the septum is generally round in plan profile, and further comprising a circumferential rib/groove interface for fluid sealing engagement with an injection port.

18. The device of claim 1, further comprising the septum in combination with an injection port housing.

19. The device of claim 15, further comprising the septum in combination with an injection port housing.

20. The device of claim 1 wherein at least one region is clear allowing human visualization of said indicia through said clear region.

21. The device of claim 19, wherein at least one region is clear allowing human visualization of said indicia through said clear region.

22. A device comprising a needle-penetrable sealing septum for an access port, comprising:
a first elastomeric region;
a second elastomeric region; and,
wherein said second region is over-molded over said first region.

23. The device of claim 22, further comprising the septum in combination with an injection port housing.

24. The device of claim 23, and further comprising indicia comprising an alpha-numeric shape embedded within the septum.

25. The device of claim 24, wherein at least one region is clear allowing human visualization of said indicia through said clear region.

26. A method of making a device comprising a needle-penetrable sealing septum for an access port, comprising:
blending medical imaging additive substantially throughout a first polymeric material to form a blend;
molding a first elastomeric region;
molding from said blend a second elastomeric region having said medical imaging additive therein;
forming indicia at least in part as an indicia void in at least one of said regions, said void occupied at least in part by the other of said regions;
wherein said medical imaging additive is substantially throughout elastomeric material in one of said regions in an amount greater than the other of said regions, providing contrast between said regions to provide visualization of said indicia.

27. The method of claim 26 wherein said second elastomeric region is over-molded over said first region after said first region is cured.

28. The method of claim 27 wherein said act of forming indicia is formed as a part of molding said first region.

29. The method of claim 26 wherein the indicia is embedded, at least partially, in the septum.

30. The method of claim 27, wherein at least one region is clear allowing human visualization of said indicia through said clear region.
